(19) United States
(12) Patent Application Publication
(10) Pub. No.: US 2008/0086109 A1
(43) Pub. Date: Apr. 10, 2008

(54) ARTERIAL CLOSURE DEVICE

(76) Inventors: Paul Shabty, Sarasota, FL (US); Rod A. Shipman, Las Vegas, NV (US); Anthony Y. Van Heugten, Sarasota, FL (US)

Correspondence Address: William H. Honaker, Esq.;DICKINSON WRIGHT PLLC, 38525 Woodward Avenue, Suite 2000, Bloomfield Hills, MI, 48304-2970, US

(21) Appl. No.: 11/942,865

(22) Filed: Nov. 20, 2007

Related U.S. Application Data
(63) Continuation-in-part of application No. 11/288,745, filed on Nov. 28, 2005.
(60) Provisional application No. 60/631,674, filed on Nov. 29, 2004.

(51) Int. Cl. A61M 31/00; A61B 17/08; A61M 29/00

(52) U.S. Cl. 604/508; 604/103.01; 606/151

(57) ABSTRACT

A method of closing an incision includes inserting a catheter (32) and the first and second balloons (50, 58) through an introducer (48) to have the first balloon (50) located in the artery (24). Next, the catheter (32) and the first balloon (50), which has been inflated, are withdrawn to apply a first force (F₁) to the puncture hole (22) and close the puncture hole (22). The method is distinguished by injecting a clotting agent (60) into a second balloon (58) to inflate the second balloon (58) and to create a cavity between the insertion hole (68) and the puncture hole (22). The slit (62) in the second balloon (58) expands into an open position as a result of a pressure (P) within the second balloon (58) to eject the clotting agent (60) therefrom. Next, the first balloon (50) is deflated, and the catheter (32) and the first and second balloons (50, 58) are removed from the insertion hole (68). Finally, a second force (F₂) is applied to the insertion hole (68) to stop the flow of blood.

### CROSS REFERENCE TO RELATED APPLICATION

This application is a continuation-in-part of U.S. patent application Ser. No. 11/288,745 filed Nov. 28, 2005, which claims priority to U.S. provisional patent application having Ser. No. 60/631,674 filed Nov. 29, 2004.

### BACKGROUND OF THE INVENTION

1. Field of the Invention

The subject invention relates to a method of closing a puncture hole in an artery, i.e., an arteriotomy site, with the arterial closure device. More specifically, the arterial closure device of the subject invention is preferably operable by a single user.

2. Description of the Prior Art

Approximately 50 years ago, the Seldinger Technique of percutaneous entry into a vascular structure by use of a needle and a guide wire technique was introduced to modem medicine and subsequently has become the standard in the medical industry. Prior to Seldinger's discovery of entry into vascular structures, procedures required an incision through the skin and tissues, followed by an incision into the artery wall.

Creating an incision through the skin, tissues, and artery wall have numerous problems associated with it, i.e., infection, uncontrolled bleeding, trauma to the tissue and vessel wall. Thus, the advent of Seldinger's Technique was widely and rapidly accepted by the medical profession, and it became the world standard due to its advantages to both patient and doctor. The patient benefited by less trauma, reduced risk of uncontrolled bleeding and vessel clotting, along with greatly reduced risk of infection. Doctors benefited by the ease of entry and exit in the procedure.

Seldinger's Technique does not require suturing the artery puncture site or the skin and adjacent tissue as earlier procedures had required. However, one main disadvantage associated with the Seldinger's Technique is that it is necessary to apply strong pressure to compress the arterial wall sufficiently to reduce blood flow and intraluminal pressure to allow initiation of the body's own hemostatic processes. Typically, compression takes between 45 minutes to one hour before closure of the arteriotomy site by natural clotting. Following this, the patient must remain inactive with bed rest for eight to twelve hours to allow the clot to strengthen. The patient often cannot return to normal activity for up to two to three days following an arteriotomy procedure.

The medical, social, and economic impact of this prolonged recovery period is considerable. In fact, with over three million arteriotomy procedures annually in just the United States, the prolonged recovery period of the Seldinger technique has an economic impact due to hospital costs incurred because of the additional day's stay. Therefore, a need exists to develop a safe and effective means for sealing the arterial wall following arteriotomy procedures that allows the patient to quickly return to normal activity.

In a recent article in the Catheter Lab Digest entitled “Vascular Access Site Hematosis: “An Endovascular Surgeon's Perspective” Manual Compression May Not Be Benign Part I, the author points out some of the problems with the manual compression on the incision site. The author discusses the incidents of access site complications that are reported as being anywhere from 0.5% to as high as 27%. However, it is known that there is no standard of reporting such complications between facilities and hospitals. Thus, these results may not mean that 27% of patients are going to the operating room to get femoral artery repairs, but they may have moderate hematomas resulting in clinical and financial expenses.

The author also reports that there are not only economic but also clinical costs to access site complications. For example, patients that have bleeding complications tend to have second stints and more have secondary events. A patient has a twelve times greater risk of dying within a year if they had bleeding complications and they are four times more likely to have other complications.

There have been other attempts to solve the problem of sealing the arteriotomy site. For example, a foreign material has been used (i.e., bovine collagen) to plug the arteriotomy site. These devices, however, rely on a non-removable biodegradable anchoring member to position the plug at the arteriotomy site. This anchoring member remains within the intraluminal space. The delayed biodegradation of the plug and its anchor can cause thrombus formation at the arteriotomy site.

Other arterial closure devices are also well known to those of ordinary skill in the art. The arterial closure devices generally comprise a body having at least one catheter with multiple ports associated with multiple lumens. The devices also generally comprise multiple balloons associated with the lumens such that one balloon closes a puncture hole in an artery, while another balloon creates a cavity adjacent the puncture hole. One of the remaining, unused ports is then used to dispense a clotting agent from the catheter to fill the cavity created by the balloon. However, one problem associated with these arterial closure devices is that multiple users are required to use these devices because of the all of the additional ports. These devices are generally used in small, tight areas where it is difficult to accommodate multiple users.

Although the prior art provides a method of closing an incision, what is desired is a method to aid in the effective and efficient deposit, in addition to the body's natural clotting agent, of additional clotting agent to the site of a puncture or small incision in the wall of a vein or artery and avoid the complications and risks of manual compression.

### SUMMARY OF THE INVENTION AND ADVANTAGES

The subject invention provides such a method by injecting a clotting agent into a second port and through a second lumen to inflate the second balloon to create a cavity between the insertion hole and the puncture hole and to create pressure on the inside of the second balloon and thus expanding a slit in the second balloon into an open position as a result of the pressure of the clotting agent within the second balloon to eject the clotting agent therefrom and to deflate the second balloon.

The subject invention provides a method that aids in the effective and efficient deposit of a clotting agent to the site of a puncture or small incision in the wall of a vein or artery. The subject invention also allows for the method to be operated by a single user. Since the arterial closure device reduces the number of additional delivery ports and because the clotting agent is effectively ejected from the second balloon, only one user is required to operate the device. Further, the subject invention avoids the complications and risks associated with manual compression techniques for closing the puncture hole.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is partial sectional view of an arterial closure device according to the subject invention;

FIG. 2 is a partial sectional, close-up view of circle 2-2 shown in FIG. 1 illustrating a first balloon in an inflated position and a second balloon in an un-inflated position;

FIG. 3 is a cross-sectional view of one embodiment of a catheter defining first and second lumens;

FIG. 4 is a cross-sectional view of another embodiment of a catheter defining first and second lumens;

FIG. 5 is a side view of an introducer inserted into an arteriotomy site having the arterial closure device adjacent thereto for insertion into the introducer;

FIG. 6 is a side view having the arterial closure device inserted into the introducer;

FIG. 7 is a side view having the first balloon in an inflated state and having the first balloon obstruct the puncture hole in the artery and having the introducer removed from the arteriotomy site;

FIG. 8 is a side view having the second balloon being inflated by a clotting agent;

FIG. 9 is a close-up side view of the first and second balloon in the inflated states and the second balloon having slits in a closed position;

FIG. 10 is a close-up side view of the slits in the second balloon in the open state having the clotting agent being ejected therefrom;

FIG. 11 is a close-up side view of the slits returning to the closed position and the second balloon and the first balloon being in the deflated state;

FIG. 12 is a close-up side view of the arteriotomy site having the arterial closure device removed therefrom and the clotting agent closing the puncture hole; and

FIG. 13 is a flow chart of an embodiment of the subject invention.

### DETAILED DESCRIPTION OF THE INVENTION

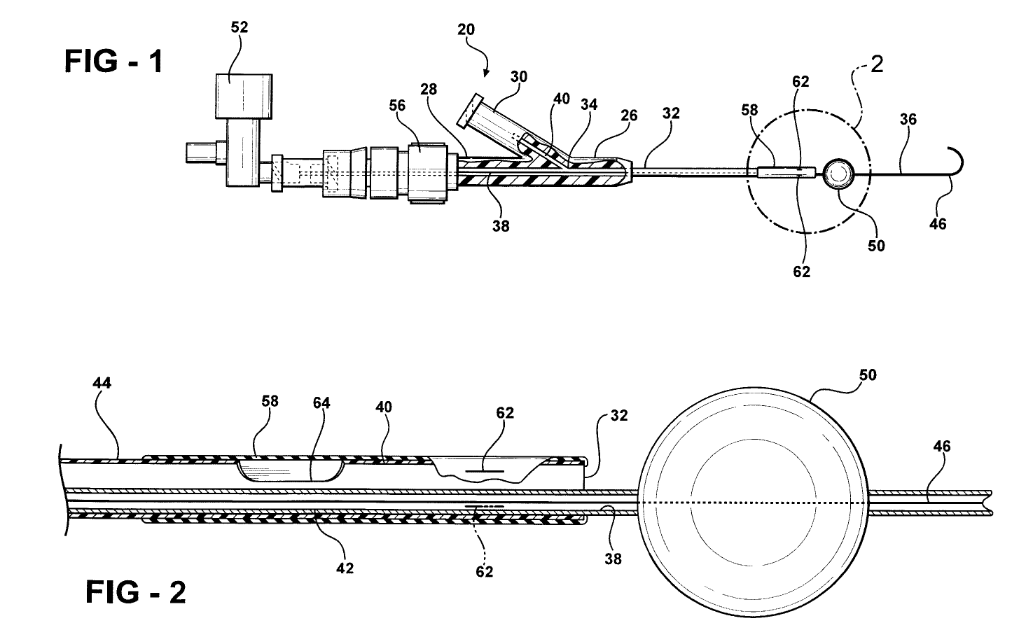

Referring to the Figures, wherein like numerals indicate corresponding parts throughout the several views, an arterial closure device is generally shown at 20 in FIG. 1. The arterial closure device 20 is particularly suited for closing a puncture hole 22 in an artery 24, generally referred to as an arteriotomy site. The arteriotomy site may result from an incision to the artery 24 or from insertion of a needle or similar medical device.

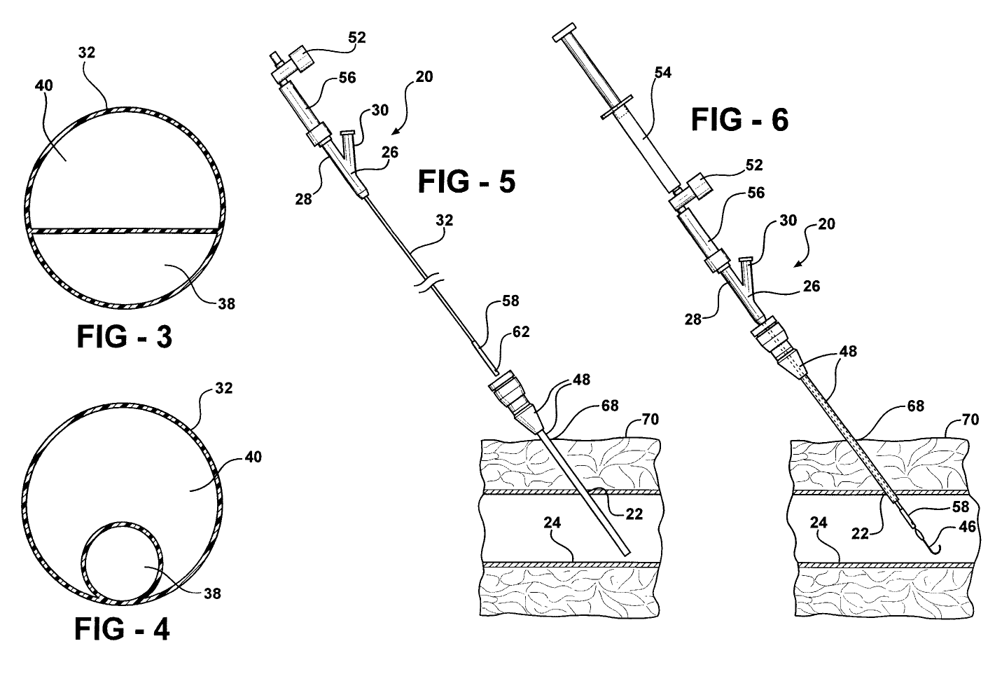

The arterial closure device 20 comprises a body 26 having a first port 28 and a second port 30 and a catheter 32 having proximal and distal ends 34, 36. The catheter 32 extends from the body 26. The catheter 32 defines a first lumen 38 in operative communication with the first port 28 and a second lumen 40 in operative communication with the second port 30. The term lumen, as defined by those of ordinary skill in the art, means a bore of a tube, such as a catheter 32. Hence, the catheter 32 can define multiple lumens 38, 40 within the bore of the catheter 32. Each of the lumens 38, 40 is generally sealed off from the other lumens 38, 40 to avoid crossover or contamination therebetween. FIG. 3 is a cross-sectional view of one embodiment of the catheter 32 defining first and second lumens 38, 40 and FIG. 4 is a cross sectional view of another embodiment of the catheter 32 defining first and second lumens 38, 40. Preferably, the two lumens 38, 40 are extruded in a round extrusion with an outer circle with two round holes inside of it, side by side.

Referring to FIG. 2, the catheter 32 may further comprise a first catheter 42 associated with the first lumen 38 and a second catheter 44 associated with the second lumen 40 such that the first and second catheters 42, 44 are separate and distinct from one another. As is generally understood by those skilled in the art, catheter 32 is intended to mean any of various tubular medical devices designed for insertion into arteries 24, canals, vessels, passageways, or body 26 cavities.

A guide wire 46 may be disposed within the catheter 32 for guiding the arterial closure device 20 into the artery 24. As appreciated by those skilled in the art, the guide wire 46 may be housed within either the first or the second lumen 38, 40 or in a separate lumen or in a separate catheter 32. Preferably, the guide wire 46 is disposed within the first lumen 38. In addition to the guide wire 46, it is common to utilize an introducer 48, shown in FIG. 5. The introducer 48 is inserted into the arteriotomy site and extends into the artery 24. Next, the arterial closure device 20 is inserted into the introducer 48 and the guide wire 46 is used to ensure proper placement within the artery 24.

Referring again to FIGS. 1 and 2, the arterial closure device 20 further comprises a first balloon 50 positioned adjacent the distal end 36 of the catheter 32 and operatively coupled to the first lumen 38 to receive a fluid through the first port 28 to expand the first balloon 50. The fluid may include any medically safe fluid to inflate the first balloon 50, such as air, saline, or the like. The first balloon 50 may have any desired shape sufficient to temporarily occluding the puncture hole 22, such as wedge shaped. Further, the first balloon 50 may be formed from any material that is capable of inflating or expanding to temporarily occlude the puncture hole 22. Examples of suitable materials include any natural or synthetic rubbers that may be used in medical procedures.

The arterial closure device 20 may also include a valve 52 operatively coupled to the first port 28 and operable between an open position and a closed position for allowing the fluid to inflate and deflate the first balloon 50. A syringe 54 (shown in FIG. 6) may be connected to the first port 28 to inject the fluid into the first balloon 50. The valve 52 may automatically close when the syringe 54 is removed to maintain pressure in the first balloon 50. A coupler 56 may be disposed between the valve 52 and the first port 28 for connecting the valve 52 to the first port 28 Alternatively, the valve 52 may directly connect to the first port 28 or the valve 52 and the coupler 56 may be integrally formed.

A second balloon 58 is spaced from the first balloon 50 a predetermined distance and operatively coupled to the second lumen 40. The predetermined distance is chosen such that when the first balloon 50 is in the inflated state, the second balloon 58 remains outside of the artery 24, i.e., extravascular, whereas the first balloon 50 is intravascular. Said another way, the predetermined distance is at least greater than the thickness of the artery 24 such that the second balloon 58 remains outside of the artery 24. Thus, it is to be appreciated by those of ordinary skill in the art that the predetermined distance can vary depending upon the size and thickness of the subject artery 24. Further, thicknesses of the artery 24 may vary with age and can be determined utilizing methods known in the art such as ultrasound or other imaging techniques. As one example, the femoral artery 24 typically has a vessel wall thickness of approximately mm, so the predetermined distance would be greater than mm.

The second balloon 58 receives a clotting agent 60, such as surgical glue, through the second port 30 to inflate the second balloon 58. The clotting agent 60 may be autologous, heterologous, or synthetic. However, any suitable clotting agent 60 may be used with the subject invention, such as Tisseel VH Fibrin Sealant. In addition to the clotting agent 60, a biologically active agent may also be eject from the second balloon 58, singly or in combination with the clotting agent 60. Suitable biologically active agents include drug cells, antibodies, anti-rejection medications, and the like. Preferably, the biologically active agent binds within the clotting agent 60 such that when the clotting agent 60 is consumed by the tissue, the biologically active agent is released. Inflating the second balloon 58 results in a cavity being formed adjacent the puncture hole 22 in the artery 24. In other words, inflation of the second balloon 58 debrides or disrupts subcutaneous tissue adjacent the artery 24 creating the cavity over the arteriotomy site for receiving a deposit of the clotting agent 60. One advantage of aggravating the tissue when using certain reactive clotting agents 60 is that tissue planes and cells are disrupted sufficiently to release tissue factor that promote conditions favorable to coagulation with the clotting agent 60.

The subject invention includes at least one slit 62 disposed in the second balloon 58. The slit 62 is expandable between an open position and a closed position in response to inflation of the second balloon 58. During the injection of the clotting agent 60, the clotting agent 60 may enter the second balloon 58 faster than it may escape causing the balloon to inflate. Alternatively, the pressure within the second balloon 58, as the clotting agent 60 is injected is low enough that the slits 62 remain in the closed position, so the second balloon 58 inflates. When the second balloon 58 is inflated and the flow of the clotting agent 60 continues, the pressure P inside the second balloon 58 expands the slit 62 from the closed position to the open position. Once the slit 62 is in the open position, the clotting agent 60 is ejected through the slit 62. The clotting agent 60 fills the distrupted extravascular cavity in the shape created by the second balloon 58, and when using certain clotting agents 60 reacts with the tissue factor to form to a tenacious, gelatinous mechanical plug that becomes firmly adhered to the artery 24 and to the tissue adjacent the artery 24 to close the puncture hole 22. The second balloon 58 elastically squeezes the clotting agent 60 through the slit 62 until the second balloon 58 deflates. As the second balloon 58 deflates, the internal pressure P within the second balloon 58 becomes sufficiently low that the slit 62 returns to the closed position.

The slit 62 has a size of from about. mm to about mm, preferably from about. mm to about mm, and most preferably from about. mm to about mm. The size of the slit 62 effects the rate that the clotting agent 60 is ejected from the second balloon 58. It is to be appreciated that the slit 62 in the open position may have various shapes, such as circular or rectangular, without being limited to any particular shape. One method of forming the slits 62 in the second balloon 58 is to pierce the second balloon 58 with a needle. However, it is to be appreciated that the slits 62 may be formed by any methods known to those of ordinary skill in the art.

Another factor in determining the rate of ejection of the clotting agent 60 from the second balloon 58 is the type of material forming the second balloon 58. Different sized slits 62 may be useable if more or less elastic materials are used to form the second balloon 58. For example, the second balloon 58 may be formed from an elastic material having an ultimate elongation of from about 50% to about 1300%. Suitable elastic materials include natural or a synthetic rubber. Preferably, the elastic material is selected from at least one of latex rubber, silicone rubber, nitrile rubber, or polyisoprene, with polyisoprene being most preferred. In addition to the type of material, a wall thickness of the second balloon 58 also impacts the rate of ejection. The second balloon 58 has a wall thickness of from about. mm to about. mm, preferably from about. mm to about. mm, and more preferably from about. mm to about. mm.

With reference to FIG. 2, the second lumen 40 has an aperture 64 to dispense the clotting agent 60 into the second balloon 58. The aperture 64 may be any shape or size so long as the clotting agent 60 is injected into the second balloon 58 under sufficient pressure P to inflate the second balloon 58. Preferably, the aperture 64 is located within the second balloon 58 and more preferably, the slit 62 is positioned downstream from the aperture 64 of the second lumen 40. As an example, it is particularly advantageous to have the slit 62 positioned from about to about mm downstream from the aperture 64 to allow adequate pressure P to inflate the second balloon 58 without opening the slit 62. Additionally, the location of the slit 62 in the second balloon 58 ensures that the clotting agent 60 remains outside of the artery 24. If the slit 62 is located on the second balloon 58 too close to puncture hole 22, the clotting agent 60 may be ejected directly into the artery 24.

Another factor contributing to the rate of ejection of the clotting agent 60 is the number of slits 62. The second balloon 58 may comprise a plurality of slits 62. The slits 62 are spaced from one another about the circumference of the second balloon 58, such that the slits 62 are axially spaced or longitudinally spaced about the circumference. Preferably, to ensure adequate ejection of the clotting agent 60, the plurality of slits 62 are spaced equally about the circumference of the second balloon 58.

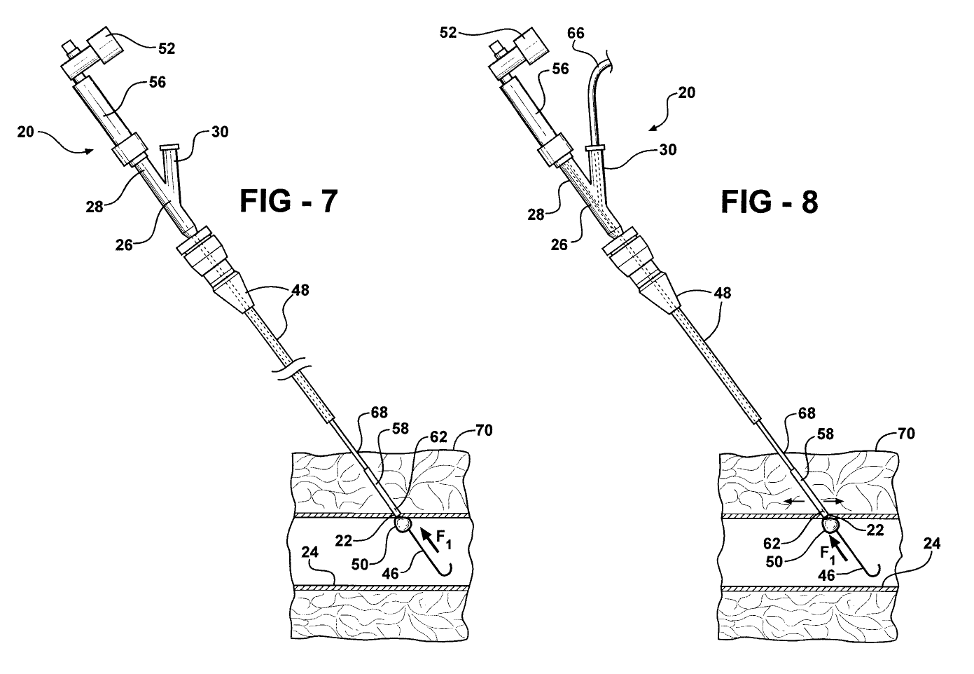

Referring to FIG. 5, the introducer 48 is inserted into the puncture hole 22. Next, the arterial closure device 20 is inserted into the introducer 48 such that the catheter 32 is inserted through the puncture hole 22 a sufficient distance to have the first balloon 50 located in the artery 24, as shown in FIG. 6. When the arterial closure device 20 is initially inserted into the artery 24, the first balloon 50 is deflated so that it can easily be inserted into the intravascular opening of the arteriotomy site. The syringe 54 is connected to the first port 28 for injecting the fluid to inflate the first balloon 50. FIG. 7 illustrates the first balloon 50 in an inflated state and the catheter 32 has been withdrawn such that the puncture hole 22 is closed with the first balloon 50. After the puncture hole 22 is closed, a delivery tube 66 is connected to the second port 30 for delivering the clotting agent 60, which is shown in FIG. 8. The delivery tube 66 may be any known device, such as single or dual tube syringe 54.

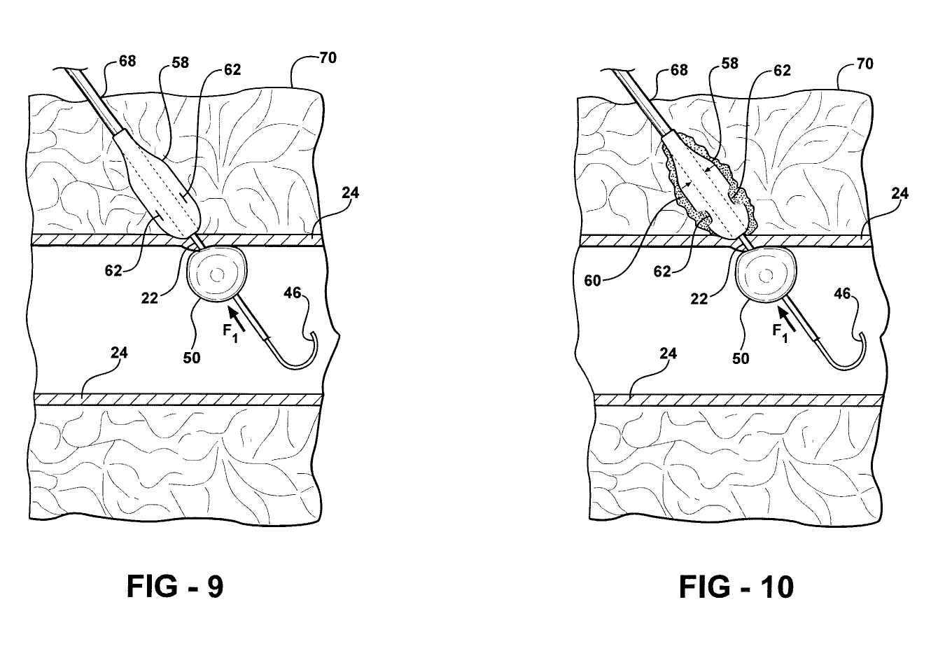

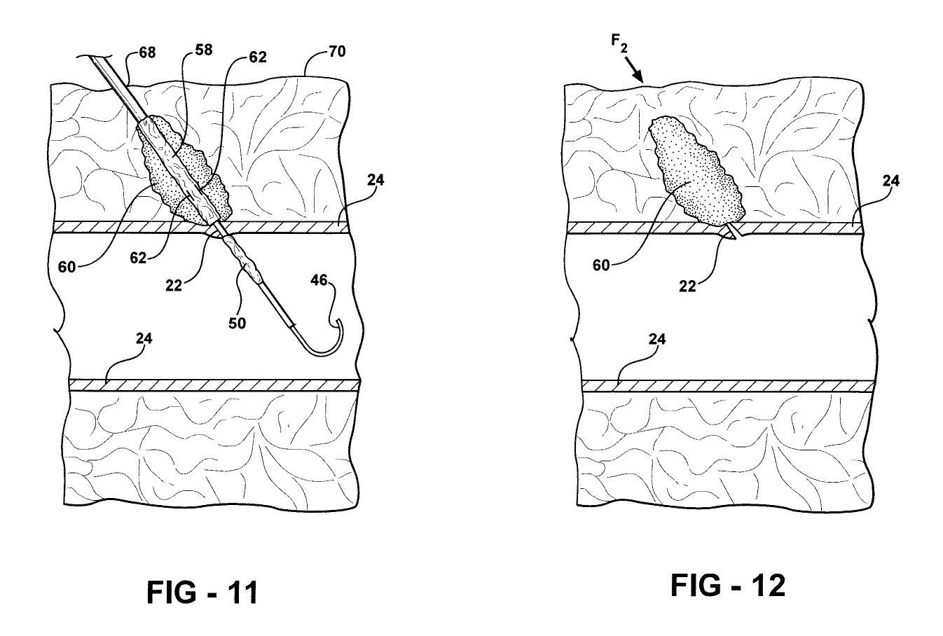

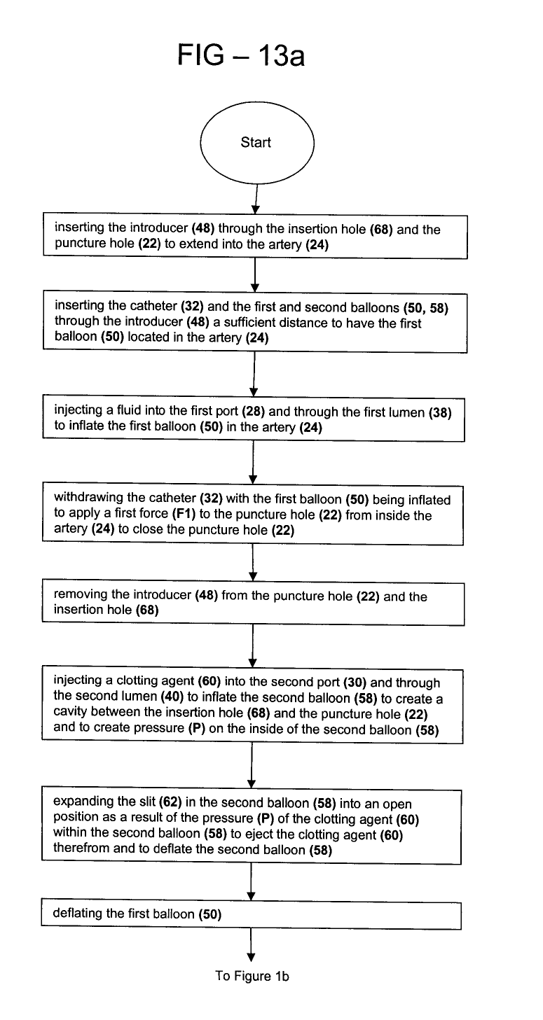

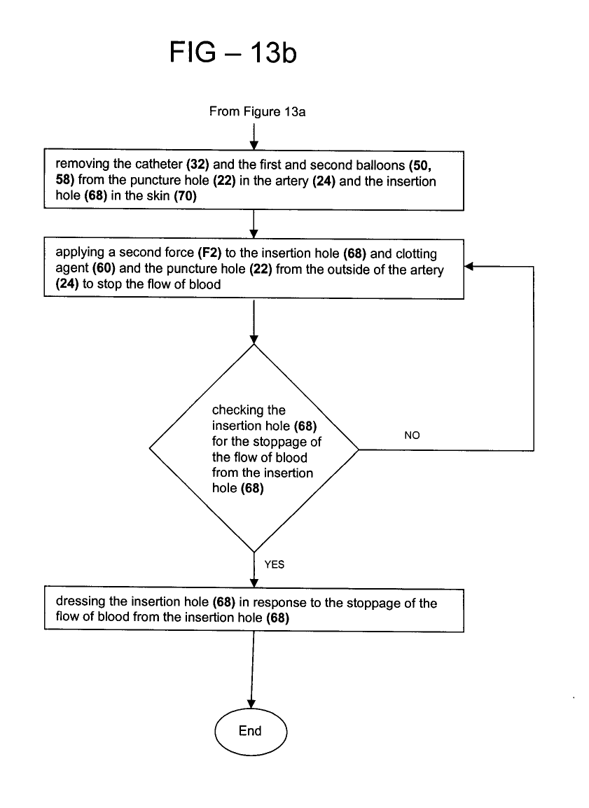

With reference to FIG. 9, the second balloon 58 has been inflated outside of the artery 24, while the first balloon 50 remains inflated. As shown in FIG. 10, the pressure P inside of the second balloon 58 has opened the slits 62 and the clotting agent 60 is being ejected from the second balloon 58. As the clotting agent 60 is ejected, the pressure P within the second balloon 58 is reduced and the slits 62 return to the closed position shown in FIG. 11. After the second balloon 58 has been deflated, the valve 52 connected to the first port 28 is again open allowing the fluid the escape from the first balloon 50, thereby deflating the first balloon 50. FIG. 12 illustrates the arteriotomy site after the arterial closure device 20 has been withdrawn and the puncture hole 22 has been closed by the clotting agent 60.

Referring back to FIG. 5, the introducer 48 is inserted through an insertion hole 68 in skin 70 and the puncture hole 22 to extend into the artery 24. As described above with reference to FIG. 6, the catheter 32 along with the first and second balloons 50, 58 are then inserted through the introducer 48 a sufficient distance to have the first balloon 50 located in the artery 24. As described above with reference to FIG. 7, a fluid is then injected into the first port 28 and through the first lumen 38 to inflate the first balloon 50 in the artery 24. The catheter 32 with the first balloon 50 being inflated is withdrawn to apply a first force F₁ to the puncture hole 22 from inside the artery 24 to close the puncture hole 22, as described above in FIG. 7. As shown in FIG. 7, the introducer 48 is then removed from the puncture hole 22 and the insertion hole 68.

As discussed above with reference to FIGS. 9 and 10, a clotting agent 60 is then injected into the second port 30 and through the second lumen 40 to inflate the second balloon 58 to create a cavity between the insertion hole 68 and the puncture hole 22 and to create pressure P on the inside of the second balloon 58. The slit 62 in the second balloon 58 expands into an open position as a result of the pressure P of the clotting agent 60 within the second balloon 58 to eject the clotting agent 60 therefrom and deflate the second balloon 58.

Next, as described above with reference to FIGS. 11 and 12, the first balloon 50 is deflated. The catheter 32 along with the first and second balloons 50, 58 are then removed from the puncture hole 22 in the artery 24 and the insertion hole 68 in the skin 70.

As shown in FIG. 12, a second force F₂ is applied to the insertion hole 68 and clotting agent 60 and the puncture hole 22 from the outside of the artery 24 to stop the flow of blood. The second force F₂ may be applied by manual compression using at least one finger or with the use of a C-clamp. In diagnostic cases, the second force F₂ is applied for about minutes five minutes. In interventional cases, the second force F₂ is applied for about fifteen minutes. After the second force F₂ has been applied to the insertion hole 68 for the desired time period above, the insertion hole 68 is checked for the stoppage of the flow of blood from the insertion hole 68. If the blood continues to flow after the second force F₂ has been applied, the second force F₂ will be reapplied until the flow of blood from the insertion hole 68 has stopped. If the flow of blood is stopped, the insertion hole 68 is dressed with gauze and bandages.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method of closing an incision having an insertion hole (68) in a skin (70) and a puncture hole (22) in an artery (24) with an introducer (48) and a catheter (32) defining first and second lumens (38, 40) in operative communication with first and second ports (28, 30) and first and second balloons (50, 58), the second balloon (58) defining at least one slit (62) therein, said method comprising;
inserting the introducer (48) through the insertion hole (68) and the puncture hole (22) to extend into the artery (24),
inserting the catheter (32) and the first and second balloons (50, 58) through the introducer (48) a sufficient distance to have the first balloon (50) located in the artery (24),
injecting a fluid into the first port (28) and through the first lumen (38) to inflate the first balloon (50) in the artery (24),
withdrawing the catheter (32) with the first balloon (50) being inflated to close the puncture hole (22),
removing the introducer (48) from the puncture hole (22) and the insertion hole (68),
injecting a clotting agent (60) into the second port (30) and through the second lumen (40) to inflate the second balloon (58) to create a cavity between the insertion hole (68) and the puncture hole (22) and to create pressure (P) on the inside of the second balloon (58),
expanding the slit (62) in the second balloon (58) into an open position as a result of the pressure (P) of the clotting agent (60) within the second balloon (58) to eject the clotting agent (60) therefrom and to deflate the second balloon (58),
deflating the first balloon (50), and
removing the catheter (32) and the first and second balloons (50, 58) from the puncture hole (22) in the artery (24) and the insertion hole (68) in the skin (70).

2. A method of closing an incision having an insertion hole (68) in a skin (70) and a puncture hole (22) in an artery (24) with an introducer (48) and a catheter (32) defining first and second lumens (38, 40) in operative communication with first and second ports (28, 30) and first and second balloons (50, 58), the second balloon (58) defining at least one slit (62) therein, said method comprising;
inserting the introducer (48) through the insertion hole (68) and the puncture hole (22) to extend into the artery (24),
inserting the catheter (32) and the first and second balloons (50, 58) through the introducer (48) a sufficient distance to have the first balloon (50) located in the artery (24),
injecting a fluid into the first port (28) and through the first lumen (38) to inflate the first balloon (50) in the artery (24),
withdrawing the catheter (32) with the first balloon (50) being inflated to apply a first force (F₁) to the puncture hole (22) from inside the artery (24) to close the puncture hole (22),
removing the introducer (48) from the puncture hole (22) and the insertion hole (68),
injecting a clotting agent (60) into the second port (30) and through the second lumen (40) to inflate the second balloon (58) to create a cavity between the insertion hole (68) and the puncture hole (22) and to create pressure (P) on the inside of the second balloon (58),
expanding the slit (62) in the second balloon (58) into an open position as a result of the pressure (P) of the clotting agent (60) within the second balloon (58) to eject the clotting agent (60) therefrom and to deflate the second balloon (58),
deflating the first balloon (50),
removing the catheter (32) and the first and second balloons (50, 58) from the puncture hole (22) in the artery (24) and the insertion hole (68) in the skin (70), and
applying a second force (F₂) to the insertion hole (68) and clotting agent (60) and the puncture hole (22) from the outside of the artery (24) to stop the flow of blood.

3. A method as set forth in claim 2 further including the step of checking the insertion hole (68) for the stoppage of the flow of blood from the insertion hole (68).

4. A method as set forth in claim 3 further comprising the step of dressing the insertion hole (68) in response to the stoppage of the flow of blood from the insertion hole (68).

5. A method as set forth in claim 2 wherein the second force (F₂) is applied by manual compression using at least one finger.

6. A method as set forth in claim 2 wherein the second force (F₂) is applied by a C-clamp.

7. A method as set forth in claim 2 wherein the step of injecting the clotting agent (60) further comprises injecting a biologically active agent with the clotting agent (60).
